(19) United States
(12) Patent Application Publication
(10) Pub. No.: US 2018/0071468 A1
(43) Pub. Date: Mar. 15, 2018

(54) RESPIRATORY AUGMENTATION DEVICE

(71) Applicant: ResMed Limited, Bella Vista (AU)

(72) Inventors: Clancy John Dennis, Potts Point (AU); Richard Charles Napper, Sydney (AU)

(73) Assignee: ResMed Limited, Bella Vista (AU)

(21) Appl. No.: 15/701,891

(22) Filed: Sep. 12, 2017

(30) Foreign Application Priority Data
Sep. 13, 2016 (AU) 2016903676

(51) Int. Cl. A61M 16/00; A61M 16/06; A61M 16/20

(52) U.S. Cl. CPC A61M 16/0069; A61M 16/0009; A61M 16/204; A61M 16/201; A61M 16/0666

(57) ABSTRACT

A respiratory augmentation device to assist or augment a user's respiration may include a pressure generator configured to generate a supply of air at positive pressure, a transducer configured to output at least one signal indicative of at least one respiratory characteristic of the user, and a controller coupled with the pressure generator and transducer. The controller may determine a cyclic pressure profile for the pressurised air and control the pressure generator to achieve the pressure profile. The controller may further determine, using the output of the at least one transducer, whether adjustment of the cyclic pressure profile is required.

### CROSS-REFERENCE TO RELATED APPLICATIONS

The present application claims priority from Australian Provisional Patent Application Number AU 2016903676, filed on Sep. 13, 2016, the entire contents of which is incorporated herein by reference.

### BACKGROUND OF THE TECHNOLOGY

### Field of the Technology

The present technology relates to one or more of the detection, monitoring, diagnosis, treatment, prevention and amelioration of respiratory-related disorders. The present technology also relates to medical devices or apparatus, and their use. More particularly, the present technology relates to a portable medical device, to assist or augment a user's respiration.

### Description of the Related Art

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide (CO₂) to move in the opposite direction. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

These can lead to reduced mobility for the patient, for example excessive sleepiness, fatigue and/or shortness of breath. The patient in some cases may experience abnormal or worsened shortness of breath on exercise. Such difficulties may lead COPD patients to avoid movement and/or exercise.

A range of therapies have been used to treat or ameliorate such conditions. Furthermore, otherwise healthy individuals may take advantage of such therapies to prevent respiratory disorders from arising. However, these have a number of shortcomings.

Various therapies, such as Continuous Positive Airway Pressure (CPAP) therapy, Non-invasive ventilation (NIV) and Invasive ventilation (IV) have been used to treat one or more of the above respiratory disorders. In some cases, supplementary oxygen (e.g. from a bottle) may be provided to the patient.

However, devices configured to provide CPAP, NIV or IV may not be suitable for some situations, such as where a user is not in bed (e.g., being mobile) and/or not sleeping. For example, a ventilator or a PAP device may not be suitable for a patient looking for alleviation of shortness of breath on exercise or during daily activities, due to its size, power requirements, cost and/or complexity. Furthermore, some devices may be designed for unconscious patients (e.g. for PAP therapy or ventilation) and may not be suitable for awake patents who breathe through their mouths, speak and/or swallow.

Bottled oxygen may not require a separate power source, however still may not be suitable due to its weight and/or size limiting portability and/or discretion. Bottled oxygen may also lack variability in terms of therapy modes.

The present technology aims to provide a solution that at least goes some way towards ameliorating or eliminating at least some of the problems of the prior art, while assisting or augmenting a user's respiration.

### BRIEF SUMMARY OF THE TECHNOLOGY

The present technology is directed towards providing medical devices used in the diagnosis, amelioration, treatment, or prevention of respiratory disorders having one or more of improved comfort, cost, efficacy, ease of use and manufacturability.

A first aspect of the present technology relates to apparatus used in the diagnosis, amelioration, treatment or prevention of a respiratory disorder.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable respiratory augmentation (‘RPA’) device that may be carried by a person, e.g., around the home of the person.

One aspect of the present technology relates to an apparatus for generating a supply of air at positive pressure for the amelioration or treatment of a respiratory disorder. The apparatus may comprise a pressure generator configured to generate the supply of pressurised air for delivery to a delivery interface of the user. The apparatus may comprise at least one transducer configured to output at least one signal indicative of at least one respiratory characteristic of the user. The apparatus may comprise a controller, coupled with the pressure generator and at least one transducer. The controller may be configured to determine a cyclic pressure profile for the pressurised air. The controller may be configured to control the pressure generator to achieve the pressure profile. The controller may be configured to determine, using the output of the at least one transducer, whether adjustment of the cyclic pressure profile is required.

According to another aspect of the present technology, the controller may be configured to determine an initial set of therapy parameters for the cyclic pressure profile.

According to another aspect of the present technology, the initial set of therapy parameters may comprise one or more of: pressure during a inspiratory portion of the cyclic pressure profile, pressure during a expiratory portion of the cyclic pressure profile, a length of the inspiratory portion of the cyclic pressure profile, and a length of the expiratory portion of the cyclic pressure profile.

According to another aspect of the present technology, the determination of the initial set of therapy parameters may be based on one or more of: user height, age, and weight.

According to another aspect of the present technology, determination of the initial set of therapy parameters may be based on a selection of one of a plurality of predetermined sets of therapy parameters.

According to another aspect of the present technology, the controller may be configured to adjust the cyclic pressure profile based at least in part on determination of at least one trigger in a breath cycle of the user.

According to another aspect of the present technology, determination of the at least one trigger may be used to control cycling of the pressure cycle.

According to another aspect of the present technology, determination of the at least one trigger may be used to control cycling at, or soon before, cessation of effort by the user.

According to another aspect of the present technology, the controller may be configured to determine the at least one trigger from one or more of: negative flow and positive flow.

According to another aspect of the present technology, the controller may be configured to distinguish triggers from false triggers.

According to another aspect of the present technology, the apparatus may comprise a pause input device, wherein the controller may be configured to pause the delivery of the supply of pressurised air on activation of the pause input device by the user.

According to another aspect of the present technology, the controller may be configured to determine the at least one trigger based at least in part on a received indication of airway openness.

According to another aspect of the present technology, the indication of airway openness may be conductance.

According to another aspect of the present technology, the controller may be configured to determine the indication of airway openness based at least in part on a measure of flow rate, and a measure of pressure.

According to another aspect of the present technology, the controller may be configured to adjust the pressure of the cyclic pressure profile based on one or more measured characteristics of the user's breath cycle.

According to another aspect of the present technology, the measured characteristics may be indicative of user effort.

According to another aspect of the present technology, the controller may be configured to adjust the pressure of the cyclic pressure profile based on at least one of: a measure of average conductance, and peak conductance, over a plurality of breath cycles.

According to another aspect of the present technology, the controller may be configured to adjust the cyclic pressure profile based on one or more predicted characteristics of the user's breath cycle.

According to another aspect of the present technology, the controller may be configured to predict a length of a next inspiratory portion of the cyclic pressure profile, and a length of a next expiratory portion of the cyclic pressure profile, based on a measured length of a previous inspiratory portion of the user's breath cycle, and a measured length of a previous expiratory portion of the user's breath cycle; and adjust the cyclic pressure profile based on the predicted length of the next inspiratory portion, and the predicted length of the next expiratory portion.

According to another aspect of the present technology, the cyclic pressure profile may comprise a first changing portion comprising a rapid increase to a high pressure, a high pressure portion, a second changing portion comprising a slower decrease from the high pressure portion, and a low pressure portion.

According to another aspect of the present technology, the controller may be configured to use a predicted length of an inspiratory portion of the cyclic pressure profile to control one or more of: timing of the transition between the high pressure portion and the second changing portion, and a rate of the decrease in pressure through the low pressure portion.

According to another aspect of the present technology, the controller may be configured to use a predicted length of an expiratory portion of the cyclic pressure profile to control an increased flow rate near the end of the expiratory portion.

According to another aspect of the present technology, the controller may be configured to control the transition between the second changing portion and the low pressure portion based at least in part of determination of at least one trigger.

According to another aspect of the present technology, the controller may be configured to interrupt control of the cyclic pressure profile based on predicted characteristics of the user's breath cycle on determination of the at least one trigger.

According to another aspect of the present technology, the controller may be configured to adjust the cyclic pressure profile on receiving a user input.

According to another aspect of the present technology, wherein the adjustment of the cyclic pressure profile on receiving the user input may comprise an adjustment of one or more of: a rate of increase in pressure through the first changing portion, a pressure level of the high pressure portion, and a hold time to maintain the high pressure portion.

According to another aspect of the present technology, the controller may be configured adjust the cyclic pressure profile to maintain the high pressure portion for one of a plurality of hold times.

According to another aspect of the present technology, the controller may be configured to select a hold time from the plurality of hold times based at least in part on whether the apparatus is in a continuous mode or an intermittent mode.

According to another aspect of the present technology, the controller may be configured to select longer hold time in the intermittent mode than in the continuous mode.

According to another aspect of the present technology, the hold time in the continuous mode may be between about 5 to 15% of an inspiratory portion of the cyclic pressure profile, and the hold time in the intermittent mode may be between about 40 to 60% of the inspiratory portion of the cyclic pressure profile.

According to another aspect of the present technology, the apparatus may comprise an oxygen port configured to allow delivery of oxygen to the user.

According to another aspect of the present technology, the oxygen port may be configured to introduce additional oxygen to the supply of pressurised air generated by the pressure generator.

According to another aspect of the present technology, the controller may be configured to control the delivery of oxygen to be at a flow rate of between 1 L/min and 7 L/min.

According to another aspect of the present technology, the controller may be configured to control the delivery of oxygen to deliver fixed concentrations of oxygen at between about 30 to 60% oxygen.

According to another aspect of the present technology, the apparatus may comprise an integral source of oxygen.

According to another aspect of the present technology, the controller may be configured to deliver a constant flow of oxygen to the user.

According to another aspect of the present technology, wherein the controller may be configured to deliver a variable flow of oxygen to the user.

One aspect of the present technology relates to a system for the amelioration or treatment of a respiratory disorder. The system may comprise a delivery interface structured and configured to deliver a supply of pressurised air to a user. The system may comprise an apparatus. The apparatus may comprise a pressure generator configured to generate the supply of pressurised air for delivery to a delivery interface of the user. The apparatus may comprise at least one transducer configured to output at least one signal indicative of at least one respiratory characteristic of the user. The apparatus may comprise a controller, coupled with the pressure generator and at least one transducer. The controller may be configured to determine a cyclic pressure profile for the pressurised air. The controller may be configured to control the pressure generator to achieve the pressure profile. The controller may be configured to determine, using the output of the at least one transducer, whether adjustment of the cyclic pressure profile is required. The system may comprise an air circuit to connect the delivery interface and the apparatus.

According to another aspect of the present technology, the delivery interface may comprise a valve configured to change a path for the pressurised air according a respiratory cycle of the user.

According to another aspect of the present technology, the valve may be configured to open a flow path between the user and the atmosphere during exhalation.

According to another aspect of the present technology, the delivery interface may be a mouthpiece interface.

According to another aspect of the present technology, the delivery interface may be a nasal interface.

One aspect of the present technology relates to a method for cycling a supply of air at positive pressure for the amelioration or treatment of a respiratory disorder. The method may comprise determining a cyclic pressure profile for the supply of pressurised air to be generated for delivery to a delivery device of a user. The method may comprise controlling generation of the pressurised air to achieve the pressure profile. The method may comprise determining, using the output of at least one transducer configured to output at least one signal indicative of at least one respiratory characteristic of the user, whether adjustment of the cyclic pressure profile is required.

The methods, systems, devices and apparatus described herein can provide improved functioning in a processor, such as of a processor of a specific purpose computer, respiratory monitor and/or a respiratory augmentation apparatus. Moreover, the described methods, systems, devices and apparatus can provide improvements in the technological field of automated management, monitoring and/or prevention and/or treatment of respiratory conditions, including, for example, COPD.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

The present technology is illustrated by way of example, and not by way of limitation, in the figures of the accompanying drawings, in which like reference numerals refer to similar elements including:

### Respiratory System and Facial Anatomy

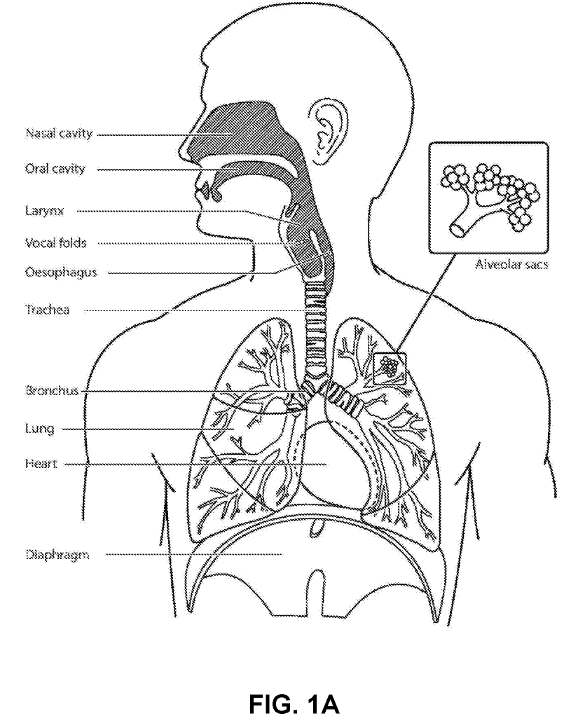

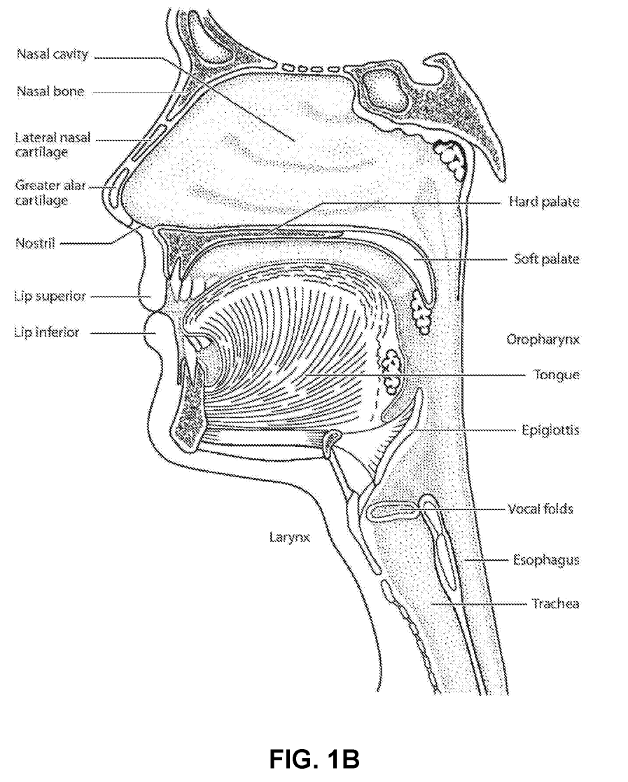

FIG. 1A shows an overview of a human respiratory system including the nasal and oral cavities, the larynx, vocal folds, oesophagus, trachea, bronchus, lung, alveolar sacs, heart and diaphragm.

FIG. 1B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

### RPA Device

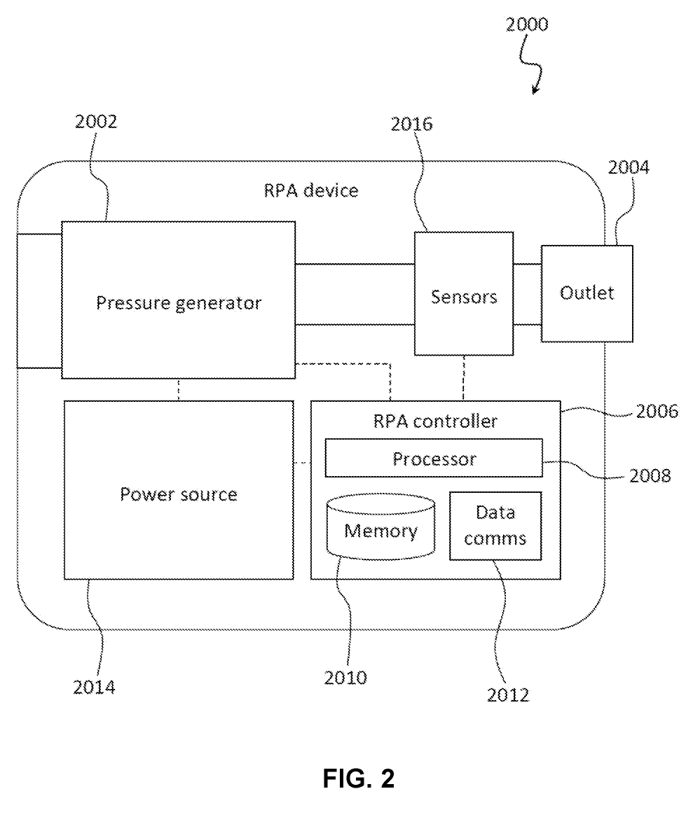

FIG. 2 shows a schematic of an RPA device in accordance with one form of the present technology.

### Delivery Interface

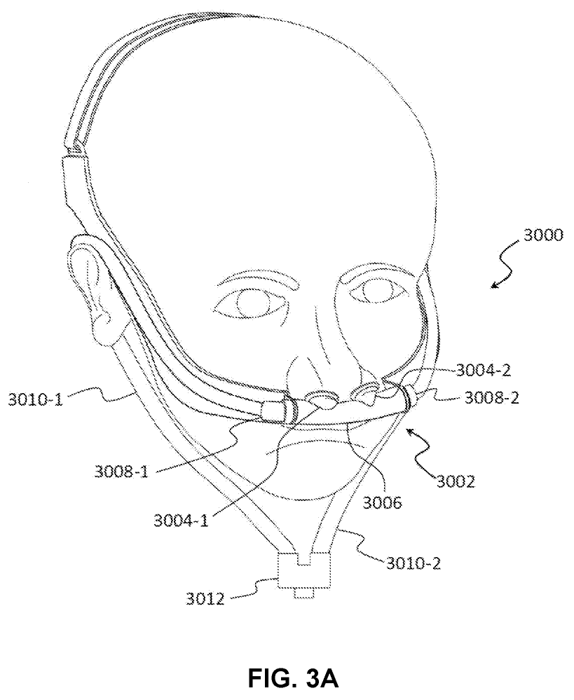

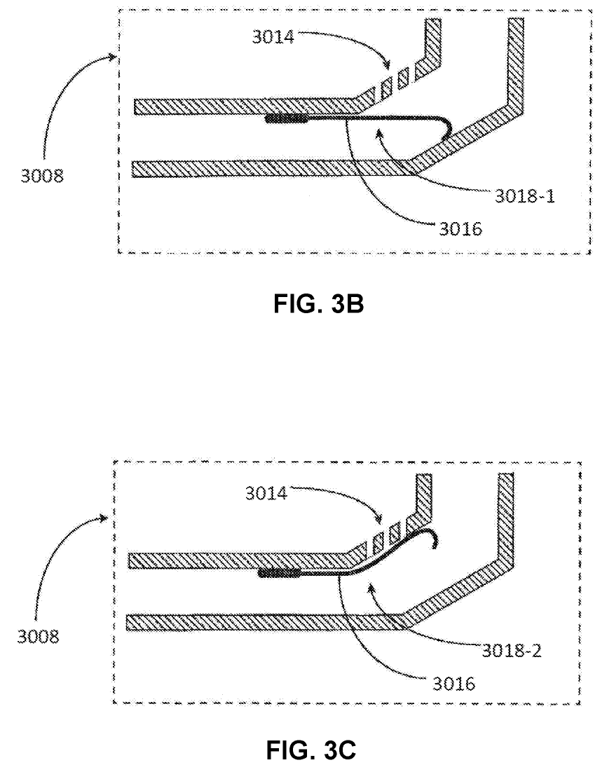

FIGS. 3A, 3B and 3C show a delivery interface in accordance with one form of the present technology.

### Breathing Waveforms

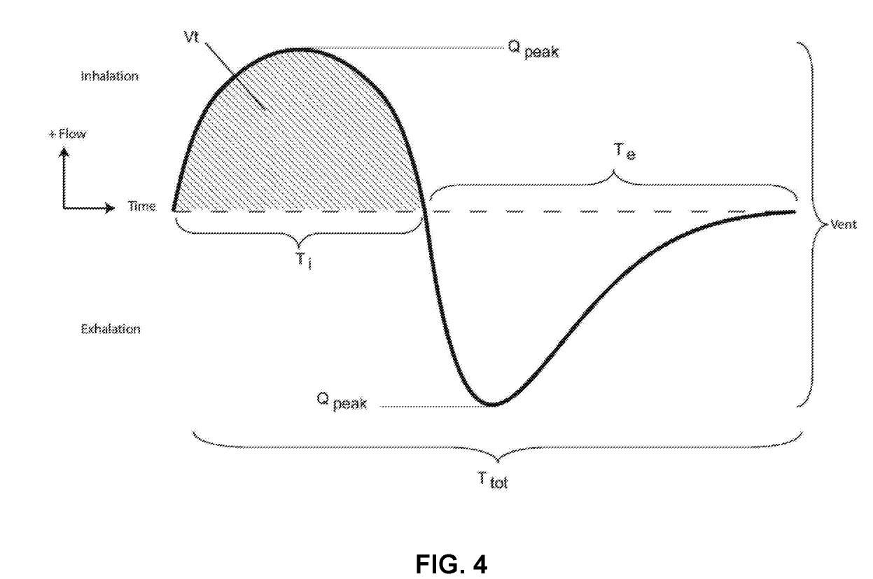

FIG. 4 shows a model typical breath waveform of a person.

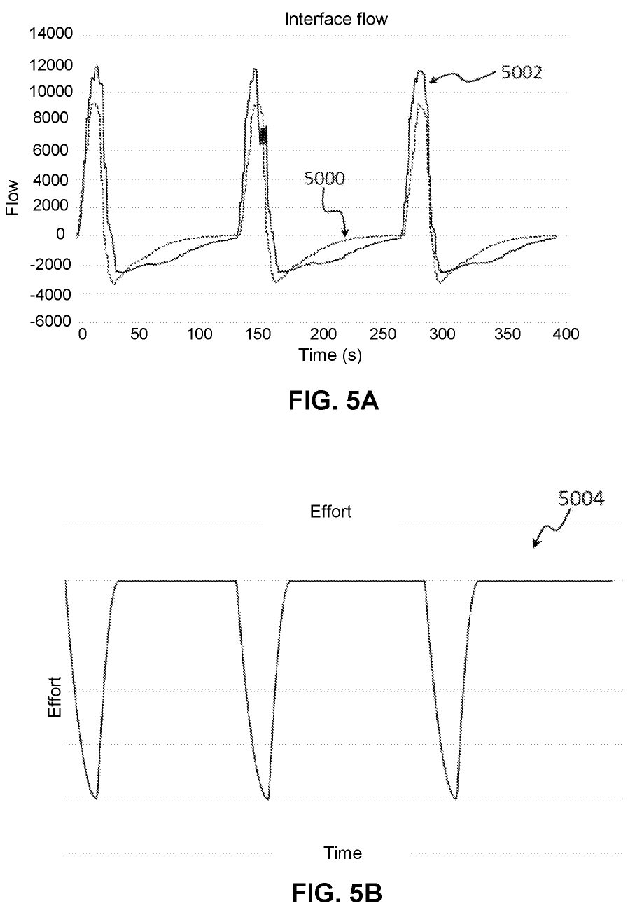

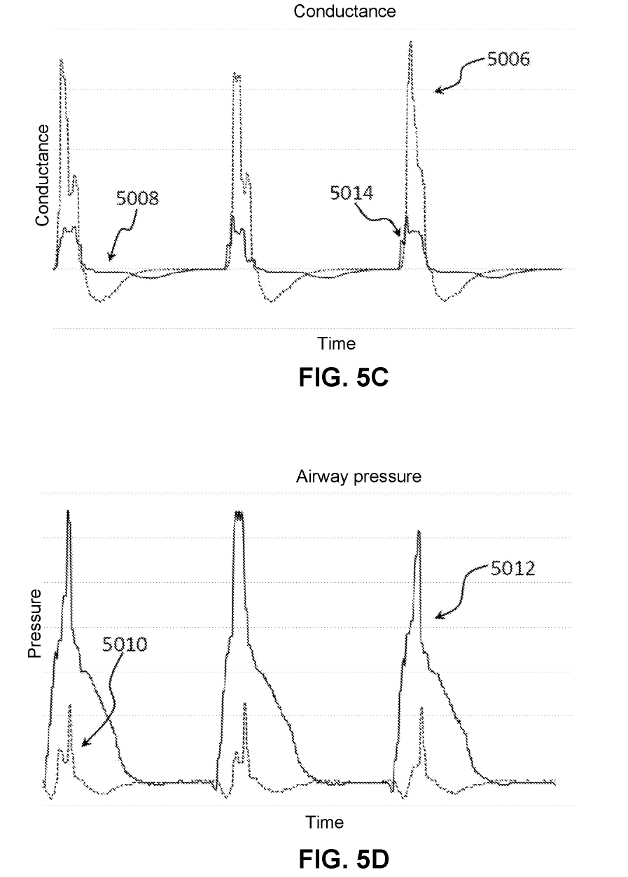

FIGS. 5A, 5B, 5C and 5D show comparative waveforms of characteristics of a person's breath both with and without respiratory augmentation in accordance with one form of the present technology.

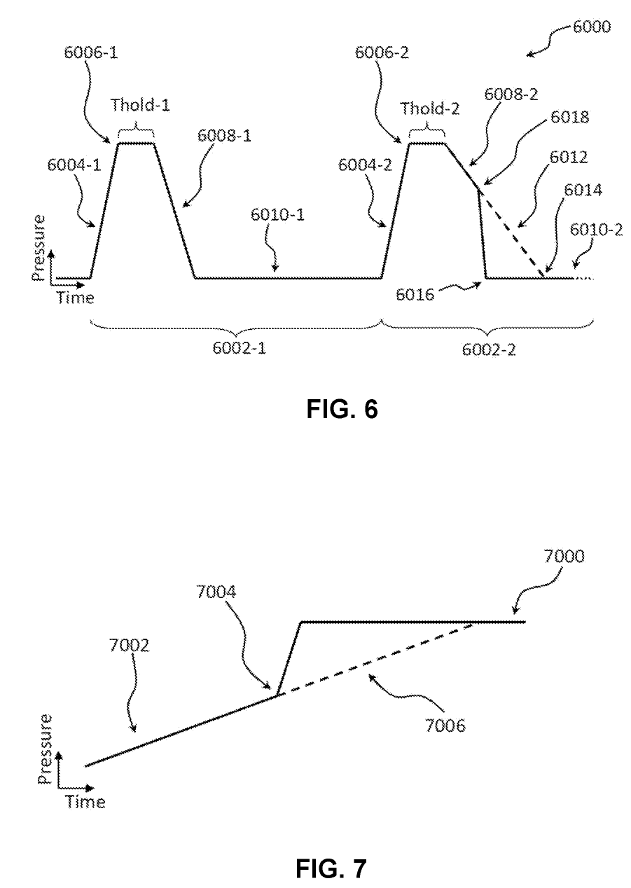

FIGS. 6 and 7 show waveforms of an RPA device in accordance with one form of the present technology.

### DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### Respiratory Augmentation

As described elsewhere in the present document, respiratory disorders may cause, or contribute to, breathlessness, or shortness of breath. For example, a COPD patient may experience breathlessness as a result of increased airway resistance with a limiting expiratory flow, decreased lung compliance and reduced ventilation/perfusion.

In some situations, this may be exacerbated, for example during exercise, or even light movement. Activity increases the generation of CO₂, in turn requiring an increase in ventilation (e.g. measured in minute ventilation, or Ve). However, COPD patients may suffer from a limited expiratory flow rate, and an increase in Ve may cause a disproportionate increase in work of breathing, after which the feeling of breathlessness ensues.

Such conditions may cause a person to become less mobile, thus leading to a cycle wherein the reduced mobility contributes to worsening of one's respiratory disorder. For instance, reduced mobility and a lack of physical activity may lead to negative physical and psychological implications, potentially resulting in faster disease progression and/or reduced fitness. Further, the sense of distress associated with breathlessness may result in anxiety and ultimately brain changes increasing sensitivity to fear.

Respiratory augmentation may assist such patients, for example to reduce the likelihood and thereby the fear and anxiety of breathlessness and allow users to exercise or perform daily activities (e.g. leaving the home, cooking and cleaning) which would normally be difficult, or even beyond their capabilities.

A review of relevant academic literature indicates that:

Inspiratory pressure support can reduce the work of breathing and increase exercise performance (Harms et al., 1997; Salvadego et al., 2015; van 't Hul et al., 2006).

Although an applied expiratory resistance has been shown to increase exercise capability it is also shown that in addition to the learned art of pursed lip breathing, people with COPD while awake may narrow their larynx reflexively to provide some internal PEEP (Baz et al., 2015).

In forms of the present technology, respiratory augmentation may reduce a user's work of breathing by increasing Vt (tidal volume) and shortening a length of time of inspiration (Ti) through pressure support. Advantageously, respiratory augmentation may shorten Ti and lengthen time of expiration (Te), thereby beneficially giving patients with expiratory obstruction more time to empty their lungs and reduce hyperinflation. Further, the ‘injecting’ of flow may assist with diffusion and reopening of airways, with larger tidal volumes at low rates increasing perfusable lung area use.

### Respiratory Device

In one form, the present technology comprises an apparatus or device for preventing or treating a respiratory disorder. The apparatus or device may comprise a respiratory augmentation (RPA) device for supplying pressurised air to the user via an air circuit to a delivery interface. It should be appreciated that reference to the device as being a RPA device is not intended to exclude its use for the treatment of a respiratory disorder.

An exemplary form of an RPA device 2000 in accordance with the present technology is illustrated in FIG. 2. In one form the RPA device 1000 may comprise a pressure generator 2002 configured to generate the supply of pressurised air for delivery to the user through an air circuit to a delivery interface via outlet 2004.

In one form the RPA device 2000 may have a controller 2006, which may comprise one or more processors 2008, memory 2010, and data communication interface 2012. Examples of a data communication interface 2012 may include WiFi, Bluetooth, Ethernet and USB. The one or more processors may be configured to implement particular control methodologies such as the algorithms or methods described in more detail herein. Thus, the controller may include integrated chips, a memory and/or other control instruction, data or information storage medium. For example, programmed instructions encompassing such a control methodology may be coded on integrated chips in the memory of the device. Such instructions may also or alternatively be loaded as software or firmware using an appropriate data storage medium.

In one form the RPA device 2000 may comprise an electrical power supply 2014, for example a battery.

In one form the RPA device 2000 may comprise one or more transducers 2016 configured to output signals indicative of one or more properties or characteristics of operation of the RPA device 2000.

The apparatus or device may be as described in any one of the following patents or patent applications the contents of which are incorporated herein by reference in their entirety: US Patent Application Publication No. 2012/0138058; and PCT Patent Application No. PCT/IB2016/051434.

### Pressure Generator

In one form the pressure generator may be a blower. The blower may be as described in PCT Patent Application Publication No. WO 2013/020167, which is incorporated herein by reference in its entirety.

In one form the pressure generator may be controlled, such as by an algorithm implemented in the controller, to generate the pressurised air having a pressure profile in accordance with one or more algorithms as described further below.

### Oxygen Delivery

It may be desirable to deliver oxygen, or supplementary oxygen to a user of the RPA device. It has been found that NIV alone may not prevent desaturation of users in later stages of COPD (Walker et al., 2015), and those users may require additional oxygen.

In one form the RPA device may comprise an oxygen port configured to allow delivery of oxygen to the user. In one form, the oxygen port may be configured to introduce additional oxygen to a flow of air generated by the device. A number of flow rates of oxygen may be suitable, such as between 1 L/min and 7 L/min, such as between 2 L/min and 6 L/min, such as 4 L/min. In another form the device might measure the incoming flow and release appropriate proportions to deliver fixed concentrations of oxygen, such as between about 30 to 60% oxygen, such as about 30% oxygen, such as about 40% oxygen, such as about 60% oxygen. In some forms, the oxygen may be delivered from a source integrated with the RPA device.

In one form the RPA device may be configured to deliver a constant flow of oxygen to the user. In one form the RPA device may be configured to deliver a variable flow of oxygen.

For example, the RPA device may be configured to provide oxygen to the user only during portions of a breath cycle, such as during inspiratory portions of a breath cycle. This may be referred to as a ‘pulsed’ delivery of oxygen, in that oxygen is not delivered to the flow of gas and/or the user when the user would not be breathing in the oxygen. In one form the RPA device may comprise a valve to synchronise the delivery of oxygen with inspiration.

### Delivery Interface

Respiratory augmentation may be delivered to a user via one of a plurality of types of delivery interface. The delivery interface may direct the air flow to a mouth of the user, or to a nose of the user, such as by forming a seal therewith. By way of example, the delivery interface may be one of: a mouthpiece interface, and a nasal interface. In some forms, the RPA device may be configured to be compatible with more than one type of delivery interface type.

A nasal interface may include any delivery interfaces configured to deliver a flow of air to an entrance to the airways via the nose. Some examples of nasal interfaces may be referred to as nasal masks (such as a ResMed Mirage FX mask or a ResMed AirFit N10 mask), or nasal pillows (such as a ResMed Swift FX nasal pillows or a ResMed AirFit P10 nasal pillows). In some forms, a nasal interface may improve accessibility of the respiratory augmentation therapy, for example in that user may wear the nasal interface even when not using the device.

In some forms, a mouthpiece interface may provide more discretion for users who wish to conceal the respiratory augmentation system when not in use.

A mouthpiece interface may include any delivery interfaces configured to deliver a flow of air to an entrance to the airways via the mouth. Some examples of mouthpiece interfaces may include those configured to be put into the mouth of the user to deliver the flow of air, and those configured to form a seal on or around a mouth of the user to deliver the flow of air. Some mouthpiece interfaces may be located away from the mouth of the user, and simply direct the flow of air to the mouth of the user.

In one form, the delivery interface may incorporate a valve configured to change a path for a flow of gas according a respiratory cycle of the user. For example, the valve may open a flow path between the user and the atmosphere during exhalation. This may reduce exhalation resistance and/or alleviate a need for the RPA device to actively provide EPAP for CO₂ ventilation. Thus, such a valve may advantageously reduce a power requirement for the RPA device, extending its operating time for a given power storage capacity.

In one form the valve may be configured to create sufficient resistance to flow that exhalation through the valve may create some EPAP pressure, for example through selection of the size of the orifice to atmosphere.

The delivery interface may be configured to allow the user to breathe in from the interface and exhale to atmosphere. In another form, the mouthpiece interface may allow the user to breathe out through the interface, such as through a valve as described above.

In one form the delivery interface, for example the nasal interface, may be non-vented, i.e. it may provide a substantially sealed flow path between the RPA device and the user during at least one phase of the respiratory cycle.

FIG. 3A-E illustrate an exemplary nasal interface 3000 and components thereof. Referring to FIG. 3A, the nasal interface 3000 comprises a cushion assembly 3002. The cushion assembly 3002 comprises a seal-forming structure in the form of nasal pillows 3004-1 and 3004-2 constructed and arranged to form a seal with a respective naris of the nose of a user, and a plenum chamber 3006. In use the plenum chamber 3006 receives the supply of air at positive pressure from the RPA device and the nasal pillows 3004-1 and 3004-2 seal with an area surrounding an entrance to the airways of the user so as to facilitate the supply of air at positive pressure to the airways. The nasal assembly further comprises flap valves 3008-1 and 3008-2, and conduits 3010-1 and 3010-2 configured to hook around the ears of a user and be connected to a connection manifold 3012 for connection to the air circuit of the RPA device. In some forms a functional aspect may be provided by one or more physical components. In some forms, one physical component may provide one or more functional aspects.

Suitable examples of flap valves 3008-1 and/or 3008-2 may include those valves disclosed in ResMed patent application WO2015/061848.

In the form of the present technology illustrated in FIG. 3B and 3C, the flap valve 3008 comprises an orifice 3014, and a moveable flap 3016 connected to an interior surface of a wall of the valve 3008 and capable of moving between a first position 3018-1 in which it extends across the fluid pathway of the valve 3008 (as illustrated in FIG. 3B), and a second position 3018-1 over the orifice 3014 (as illustrated in FIG. 3C).

As illustrated in FIG. 3C, during the inspiratory portion of a breathing cycle, the flap 3016 is sealed over the orifice 3014 by the inflow of air towards the cushion assembly 3002. Conversely, FIG. 3B shows the flap 3016 during the expiratory portion of a breathing cycle, sealing against an opposing surface of the valve 3008 and directing air flow through the orifice 3014.

### Transducers

In one form of the present technology the transducers may be internal of the RPA device, or external of the RPA device. One or more transducers may be constructed and arranged to measure properties such as a flow rate, a pressure or a temperature at any point in the pneumatic path. Such transducer(s) may include, for example, a flow rate sensor (flow sensor) such as a differential pressure transducer or hot wire sensor, a pressure sensor, a temperature sensor, etc.,

In one form of the present technology, one or more transducers may be located proximate to the delivery interface.

In one form of the present technology one or more transducers may be configured to detect movement, for example an accelerometer based sensor. Such movement detection may be used as an indication of user activity while using the RPA device. This information may be used, for example, as an indicator of user acceptance of the RPA device, or as a gauge of the effectiveness of the use of the RPA device over time.

In one form, the indication of user activity may be used to anticipate the potential level of demand for augmentation by the user—for example a higher level of activity may require a greater sensitivity to conditions of breathlessness.

### Treatment Algorithms

FIG. 4 shows a model typical breath waveform of a person, such as while sleeping or awake. The horizontal axis is time, and the vertical axis is respiratory flow rate. While the parameter values may vary, a typical breath may have the following approximate values: tidal volume, Vt, 0.5 L, inhalation time, Ti, 1.6 s, peak inspiratory flow rate, Qpeak, 0.4 L/s, exhalation time, Te, 2.4 s, peak expiratory flow rate, Qpeak, −0.5 L/s. The total duration of the breath, Ttot, is about 4 s. The person typically breathes at a rate of about 15 breaths per minute (BPM), with Ventilation, Vent, about 7.5 L/minute. A typical duty cycle, the ratio of Ti to Ttot is about 40%.

Respiratory augmentation therapy may comprise assisting a user to take in a short voluminous inspiratory breath (i.e. a high Q over a short Ti to achieve a desired Vt) accompanied by an unhindered and relaxed expiratory breath, by supplying a positive pressure during an inspiratory portion of a breath, while providing zero pressure during an expiratory portion of a breath. Respiratory augmentation therapy is envisaged to reduce respiratory rate, dynamic hyperinflation, the respiratory effort required to achieve sufficient ventilation, and the sensation of discomfort associated with burdened breathing.

In one form, an initial set of therapy parameters (e.g. initial therapy target) for the respiration augmentation therapy may be determined for a user. In one form the initial therapy target may be determined based on, for example, one or more of: user height, age, and weight inputs.

For example, one or more of: pressure to be provided during the inspiratory, or expiratory portion of a breath, or a length of an inspiratory or expiratory portion of a breath, may be determined at least partly based on the user height, age and weight inputs. In one form, the initial therapy target may be selected from a plurality of predetermined initial therapy targets having relatively higher or lower parameter levels, for example: low, medium, and high.

It is envisaged that by providing initial therapy targets, previously determined to be suitable to the input characteristics, the likelihood of initial user comfort and acceptance of the RPA device may be increased. A user's first experience of the RPA device may influence their willingness to maintain use—whether the initial use be ineffective (i.e. the initial therapy target is perceived as being too low for the user), or perceived as being overly aggressive (i.e. the initial therapy target is perceived as being too high for the user).

In one form, the RPA device may determine an estimate of energy expenditure based on these inputs. In one form the RPA device may output an indication of the estimate of energy expenditure—for example via a display device on the RPA device, or transmitted to another user device.

### Triggering

In one form, operation of the RPA device may comprise a triggering algorithm. The triggering algorithm may determine one or more events or occurrences in a breath cycle of a user—i.e. triggers. Such events or occurrences may be one or more feature detected from a signal(s) of a transducer(s) or other sensor described herein (e.g., a flow sensor and/or pressure sensor). The RPA device may be configured to control the delivery of pressurised air to the delivery interface based at least in part on determination of at least one trigger in a breath cycle.

In one form, determination of the trigger may be implemented to control changes of the delivery of pressurised air, which may be referred to herein as “triggering”. It may be desirable for triggering to be responsive in order to increase the extent of inspiratory pressure support, particularly in the initial stages of inspiration.

Expiratory work imposed due to late cycling (e.g., late reduction in delivered pressure) may decrease the effectiveness of the respiratory augmentation therapy. In one form, one or more triggers may be implemented to control cycling at, or soon before, cessation of effort by the user. The RPA device may be configured to reduce, or cease, delivery of pressurised air at this time for user comfort, and to reduce the likelihood of expiratory muscle activation occurring.

In one form, determination of the at least one trigger may be made from negative flow (e.g., a flow rate indicative of expiratory flow). In one form the determination of the at least one trigger may be made from negative flow or positive flow (e.g., a flow rate indicative of expiratory flow or a flow rate indicative of inspiratory flow).

In one form the RPA device may be configured to distinguish triggers from false triggers. Examples of such false triggers may include one or more of: speech, swallowing, and opening of the mouth.

In one form the RPA device may comprise a pause input device such as a toggle switch, activation of which by the user may pause the operation of the RPA device to allow for an occurrence which may otherwise act as a false trigger (for example, speech, swallowing, and opening of the mouth). By way of example, the toggle switch may be a depressible button, a software button selectable through a touch screen, or a selectable switch.

In one form, the triggering algorithm may be configured to use an indication of airway openness as an input. In one form, the indication of airway openness may be conductance.

In one form, a triggering algorithm may receive as inputs a measure of flow rate, and a measure of pressure to determine the indication of airway openness. In one form the measure of flow rate may be of positive flow rate.

In one form conductance (Gmeas) may be calculated, either continuously or periodically, using the following equation:

$${G}_{meas} = \frac{{Q}^{1.75} \times {ρ}^{0.75}}{Δ P}$$

Where the symbols indicate:
Q=delivery interface flow rate
Δ=Airway pressure
ρ=Outlet gas density

Such a measure utilises a change in user conductance as the user inhales or exhales. This may assist in determination of triggers, such as a conductance trigger, even when a negative flow condition exists, and/or when a user is breathing through their mouth while connected to a nasal interface provided the soft palate connecting the nose and mouth is open.

Conductance-based cycling (i.e., evaluating conductance as an input/test for triggering one or more changes in delivered pressure (e.g., ceasing an increase in inspiratory pressure and/or decreasing an inspiratory pressure)) may assist in triggering a pressure change (e.g., decrease) at the peak point of the inspiratory effort, at which point there might still exist a non-zero inspiratory flow. The reduction then relaxation of muscular effort produces a decrease in conductance. For example, a calculated conductance, such as previously described, may be compared to one or more thresholds for making one or more changes to inspiratory pressure. Thus, in some cases, such pressure(s) may be set as a function of a continuously or periodically calculated conductance.

### Pressure Support

As described elsewhere, the RPA device may be configured to modify a user's breath profile to reduce a time length of inspiration (Ti), and increase a tidal volume (Vt) of the user. The RPA device may comprise a pressure support profile configured to assist a user to do so. Thus, the controller of the RPA may be configured, e.g., with programming of the processor to control changes in speed of a motor of the blower of the RPA, to control generation of delivered pressure to substantially conform to any of the profiles described herein.

Such a breath profile may beneficially reduce an end expiratory lung volume by extending time available to empty the lungs, thereby reducing a demand for expiratory muscle activation. For a user who may be suffering from a condition such as COPD, this may reduce a requirement for the user to actively exhale.

FIG. 5A to 5D show an exemplary set of waveforms of characteristics of a user's breath cycle, with and without augmentation by the RPA device. FIG. 5A shows exemplary breath waveforms of a user in terms of flow rate at the delivery interface against time. A first breath waveform 5000 illustrates flow rate without assistance from the RPA device, while a second breath waveform 5002 illustrates flow rate with augmentation by the RPA device. FIG. 5B shows the user's effort waveform 5004—noting that the user's effort remains the same for both with and without augmentation, but the effectiveness of that effort is greater when augmented. FIG. 5C shows the conductance of the user in a first conductance waveform 5006 without augmentation, and a second conductance waveform 5008 with augmentation. FIG. 5D shows the airway pressure of the user in a first pressure waveform 5010 without augmentation, and a second pressure waveform 5012 with augmentation.

It may be seen in FIG. 5A that the flow rate of the second breath waveform 5002 achieves a greater peak flow, and more quickly, than the first unassisted breath waveform 5000—leading to a shortened Ti and increased Vt.

From comparison of the first conductance waveform 5006 with the second conductance waveform in FIG. 5B it may be seen that conductance is significantly reduced with augmentation by the RPA device. The trigger conductance may be seen in the second conductive waveform 5006, for example at trigger point 5014. Following triggering, conductance increases as the result of pressure supplied by the RPA device (see, for example, second pressure waveform 5012 of FIG. 5D) until the inflection in muscle effort as the top of the pressure/volume lung sigmoid is reached.

In one form, air flow delivered by the RPA device over a breath may comprise a pressure profile characterised by a first changing portion comprising a rapid increase to a high pressure, a high pressure portion, a second changing portion comprising a slower decrease from the high pressure portion and a low pressure portion.

In one example, the RPA device may deliver an air flow over one or more breaths, the air flow comprising a pressure profile 6000 as shown in FIG. 6. The pressure profile 6000 shows a first breath cycle 6002-1 and a second breath cycle 6002-2. The first breath cycle 6002 shows a first changing portion 6004-1 comprising a linear increase in pressure, a high pressure portion 6006-1 comprising a constant pressure maintained over a hold time (Thold-1), a second changing portion 6008-1 comprising a linear decrease in pressure, and a low pressure portion 6010-1 comprising a constant pressure.

The rapid increase in pressure through the first changing portion 6004-1, and maintaining it for a time Thold-1 through the high pressure portion 6006-1 may assist in augmenting the user's inspiration to encourage a reduced Ti. The subsequent decrease in pressure through second changing portion 6008-1 may reduce the likelihood of the air flow resisting expiration of the user, thereby encouraging an increase in Te and reducing end expiratory lung volume.

In one form, an initial interface pressure (for example, the pressure at the high pressure portion 6006-1 to be delivered to a respiratory interface) may be determined based on a target flow rate, wherein the target flow rate is calculated from one or more user characteristics or settings.

In one form, an updated interface pressure may be determined based on one or more measured characteristics of the user's breath cycle. In one form the measured characteristics may be indicative of user effort, in order to account for changes in the user's conditions or respiratory demand.

In one form, the updated interface pressure may be determined based on at least one of: measures of average conductance, and peak conductance, over multiple breaths. By tracking the changes in conductance the relative changes in user effort may be estimated and used to increase or decrease the setting for the interface pressure.

As user effort increases and the pressure in the mouth drops, the flow for a given interface pressure will increase. This may cause the average measured conductance to increase and the updated interface pressure to increase. As interface pressure increases, flow from the RPA device may increase and effort from the user may decrease. Flow may then decrease and the calculated conductance will tend back towards the original value allowing a relative equilibrium to be set with automated proportional control of pressure to respiratory effort.

In one form the pressure profile may be adjusted based on one or more predicted characteristics of the user's breath cycle.

In one form, Ti and Te may be tracked for each breath cycle, and used to predict the Ti and Te expected for the next breath cycle. For example, Ti-predicted may be determined using the following formula:

Ti-predicted=(Ti-predicted+Ti-meas)/2

Where Ti-meas is the measured time of the inspiration of the previous breath.

Similarly, Te-predicted may be determined using the following formula:

Te-predicted=(Te-predicted+Te-meas)/2

Where Te-meas is the measured time of the expiration of the previous breath.

In one form, the Ti and Te of a first breath cycle may be determined based on an initial therapy target for the respiration augmentation therapy for the user. These initial target values may act as the predicted values in the formula described above until a second cycle has been performed.

In one form, the RPA device may implement the Ti-predicted to control the timing of the transition between the high pressure portion 6006-1 and the second changing portion 6008-1, and/or the rate of the decrease in pressure to the low pressure portion 6010-1.

In one form, the RPA device may implement the Te-predicted to increase flow near the end of expiration to increase trigger sensitivity and ready the pressure generator for operation (for example, readying a motor of a blower for acceleration).

In one form, a triggering algorithm substantially as described above may be used to control the transition between the second changing portion (e.g., 6008-1) and the low pressure portion (e.g., 6010-1). In one form triggering may interrupt control of the pressure profile based on the predicted characteristic of the breath cycle, for example one of: Ti-predicted and Te-predicted.

By way of example, in FIG. 6 it may be seen that the pressure profile of the second breath cycle 6002-2 has been adjusted from the first breath cycle 6002-1 based on Ti-predicted and Te-predicted. The predicted slope for the second changing portion 6008-2 is shown in dashed line 6012, through to a predicted cycle point 6014 for transition to the low pressure portion 6010-2.

However, the conductance trigger may occur earlier than the Ti-predicted on which the pressure profile is based. Through detection of this at cycle point 6016 using the trigger algorithm's monitoring of Gmeas (e.g., a comparison of calculated conductance and a threshold), a pressure profile adjustment 6018 may be performed to shorten the predetermined decay of pressure to the second changing portion 6010-2. This may assist in realignment of the cycling of the pressure profile with the breath cycle of the user.

### 5.3.3 User Adjustment of Pressure Profile

In one form, the RPA device may be configured to adjust the pressure profile on receiving a user input. For example, the RPA device may comprise a user input device such as a toggle switch. By way of example, the toggle switch may be a depressible button, a software button selectable through a touch screen, or a selectable switch.

In one form, the user adjustment may be an increase in one or more of: the rate of increase through the first changing portion (e.g., 6004-1), the high pressure value (e.g., 6006-1), and the hold time (Thold) of the high pressure portion.

It is envisaged this increase may provide accelerated respiratory augmentation to allow a selectively large breath—for example when preparing to undertake a challenging task, or action such as a cough which may induce breathlessness, or generally if the user considers that augmentation is not reacting fast enough to rising dyspnoea. The increase may assist the user in overcoming the challenge of initial acceptance of use of the RPA device, and/or some patients may have a higher than anticipated demand for support.

In one form the increase may be performed on receiving a single selection of the user input device, for a predetermined period of time. In one form the increase (or at least a maximum thereof) may be maintained during selection of the user input device—i.e. until it is released.

In one form the increase may be controlled based at least in part on the initial therapy target, or inputs used to determine same.

FIG. 7 shows a pressure profile 7000 having a first changing portion 7002 comprising a constant increase at a first rate to a high pressure Ptarget. On receiving a user input at time 7004, the pressure profile is adjusted to increase at a second rate higher than that of the first rate until Ptarget is achieved. The dashed line 7006 illustrates the time difference in the Ptarget being achieved.

### Delivery Interface-Specific Algorithms

A RPA device may be configured to be operable with any of a plurality of respiratory delivery interfaces. In one form, the RPA device may receive a delivery interface type as an input, whereupon one or more parameters of the air flow delivered to the user may be determined.

For example, the RPA device may comprise a delivery interface type selection device such as a toggle switch to cycle through a plurality of predetermined delivery interface types. By way of example, the toggle switch may be a depressible button, a software button selectable through a touch screen, or a selectable switch.

In one form the RPA device may be configured to determine the type of respiratory delivery interface in use, and select the mode of operation based at least in part on the determination of the type of delivery interface.

As examples of delivery interface type specific mode of operation algorithms, a ‘continuous’ mode and an ‘intermittent’ mode are described below.

### Continuous Mode

In one form of the present technology, the RPA device may be configured to operate in a mode adapted for continuous use (‘continuous mode’)—i.e. where the respiratory delivery interface is configured to be in continuous communication with the user's airways. It is envisaged that forms of nasal interfaces may be more suited to continuous use than other interfaces in terms of discretion and comfort. Nevertheless, in some forms, mouthpiece interfaces may be used in the continuous mode.

At rest, breathing may be primarily through the nasal passage, possibly due to the inherit conditioning and filtering properties. As relative effort and metabolic work increases the oral passage is opened and oronasal ventilation begins. The opening of the mouth has the effect of roughly halving the inspiratory resistance.

According to studies, the transition point to oronasal ventilation is 35+/−10 LPM VE at which point 57% of total ventilation is nasal. At 45 LPM, nasal ventilation is ˜50% of totally ventilation and then drops to 39% at peak ventilation (Niinimaa et al., 1980; Rodenstein and St\check{a}nescu, 1984; Wheatley et al., 1991). Applying pressure at the nares overcomes the resistance, increasing flow. It should be appreciated that relative nasal resistance and user effort may dictate the actual flow for a given interface pressure.

In one form, in the continuous mode the RPA device may operate in a manner substantially as described above.

It is envisaged that the adjustment of the pressure profile based on user initiated input may have particular application to the continuous mode.

### Intermittent Mode

In one form of the present technology, the RPA device may be configured to operate in a mode adapted for use with a respiratory delivery interface configured to be intermittently accessed by a user (‘intermittent mode’).

In one form the intermittent mode may be configured to operate to prevent breathlessness, in a manner substantially as described above—for example with reference to the continuous mode. In one form the intermittent mode may be configured to operate to treat breathlessness.

In some examples of the present technology, an RPA device may be configured to provide a flow of air to the user at one of a plurality of hold times, such as either of a first hold time or a second hold time, wherein the first hold time is longer than the second hold time. Furthermore, the RPA device may be further configured to provide one of the plurality of hold times based at least in part on a type of interface that the RPA device is connected to.

In one form, in the intermittent mode the RPA device may be configured to provide a longer hold time (Thold) of a high pressure portion of a pressure profile substantially as described previously, in comparison with a Thold of the continuous mode. In one form a longer Thold may be provided when the intermittent mode is used to treat breathlessness. It is envisaged that such a longer Thold may act as a “sigh” breath (i.e. a comparatively large filling of the lungs to reopen parts of the lung and encourage diffusion—like a sigh). The intermittent mode is envisaged to being better suited to such a longer Thold due at least in part to the ability of the user to remove the interface before exhaling, thereby reducing the need to manage cycling for comfort purposes in comparison with the continuous mode.

By way of example, in one form Thold may be about 5 to 15% of inspiratory time in the continuous mode, and about 40 to 60% of the inspiratory time in the intermittent mode. In one form Thold may be about 10% of inspiratory time in the continuous mode, and about 50% of the inspiratory time in the intermittent mode.

### GLOSSARY

For the purposes of the present technology disclosure, in certain forms of the present technology, one or more of the following definitions may apply. In other forms of the present technology, alternative definitions may apply.

### General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT or RPA device or emanating from a mask or delivery interface. Ambient noise may be generated by sources outside the room.

Conductance: A measure of an ability of air to flow through a conduit, air passage, or respiratory tract. Conductance may be given the symbol G.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’.

Leak: The word leak will be taken to be an unintended flow of air. In one example, leak may occur as the result of an incomplete seal between a mask and a patient's face. In another example leak may occur in a swivel elbow to the ambient.

Patient: A person, whether or not they are suffering from a respiratory condition.

Delivery Interface: A delivery interface may be used to interface respiratory equipment to its wearer, for example by providing a flow of air to an entrance to the respiratory airways. The flow of air may be provided via a mask to the nose and/or mouth, a tube to the mouth or a tracheostomy tube to the trachea of a patient. Depending upon the therapy to be applied, the delivery interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the delivery interface may not include a seal sufficient to facilitate delivery to the airways of a supply of gas at a positive pressure of about 10 cmH₂O.

### Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### Respiratory Cycle

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Inspiratory portion of a breathing cycle: The period from the start of inspiratory flow to the start of expiratory flow will be taken to be the inspiratory portion of a breathing cycle.

Positive End-Expiratory Pressure (PEEP): The pressure above atmosphere in the lungs that exists at the end of expiration.

Tidal volume (Vt): The volume of air inhaled or exhaled during normal breathing, when extra effort is not applied.

(inhalation) Time (Ti): The duration of the inspiratory portion of the respiratory flow rate waveform.

(exhalation) Time (Te): The duration of the expiratory portion of the respiratory flow rate waveform.

### Ventilation

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath. If the pressure waveform template □(□) is zero-valued at the end of expiration, i.e. □(□)=0 when □=1, the EEP is equal to the EPAP.

Inspiratory positive airway pressure (IPAP): Maximum desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS =IPAP−EPAP). In some contexts pressure support means the difference which the ventilator aims to achieve, rather than what it actually achieves.

(hold) Time (Thold): The duration for which a high pressure portion of a pressure profile for pressure support is maintained.

Triggered: When a ventilator delivers a breath of air to a spontaneously breathing patient, it is said to be triggered to do so at the initiation of the respiratory portion of the breathing cycle by the patient's efforts.

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### REFERENCE SIGNS LIST

### Citations

### Patent Literature

### Non-Patent Literature

Baz, M., Haji, G. S., Menzies-Gow, a., Tanner, R. J., Hopkinson, N. S., Polkey, M. I., Hull, J. H., 2015. Dynamic laryngeal narrowing during exercise: a mechanism for generating intrinsic PEEP in COPD? Thorax 70, 251-257. doi:10.1136/thoraxjn1-2014-205940

Harms, C. A., Babcock, M. A., McClaran, S. R., Pegelow, D. F., Nickele, G. A., Nelson, W. B., Dempsey, J. A., 1997. Respiratory muscle work compromises leg blood flow during maximal exercise. J. Appl. Physiol. 82, 1573-83.

Niinimaa, V., Cole, P., Mintz, S., Shephard, R. J., 1980. The switching point from nasal to oronasal breathing. Respir. Physiol. 42, 61-71. doi:10.1016/0034-5687(80)90104-8

Rodenstein, D. O., St\check{a}nescu, D. C., 1983. Absence of nasal air flow during pursed lips breathing. The soft palate mechanisms. Am. Rev. Respir. Dis. 128, 716-718.

Rodenstein, D. O., St\check{a}nescu, D. C., 1984. Soft palate and oronasal breathing in humans. J. Appl. Physiol. 57, 651-657.

Salvadego, D., Sartorio, A., Agosti, F., Tringali, G., Patrizi, A., Mauro, A. Lo, Aliverti, A., Grassi, B., 2015. Acute respiratory muscle unloading by normoxic helium-O2 breathing reduces the O2 cost of cycling and perceived exertion in obese adolescents. Eur. J. Appl. Physiol. 115, 99-109. doi:10.1007/s00421-014-2993-8

van 't Hul, A., Gosselink, R., Hollander, P., Postmus, P., Kwakkel, G., 2006. Training with inspiratory pressure support in patients with severe COPD. Eur. Respir. J. 27, 65-72. doi:10.1183/09031936.06.00036505

Walker, D. J., Walterspacher, S., Ekkernkamp, E., Storre, J. H., Windisch, W., Dreher, M., 2015. Walking with Non-Invasive Ventilation Does Not Prevent Exercise-Induced Hypoxaemia in Stable Hypercapnic COPD Patients. COPD. doi:10.3109/15412555.2015.1008690

Wheatley, J. R., Amis, T. C., Engel, L. A., 1991. Oronasal partitioning of ventilation during exercise in humans. J. Appl. Physiol. 71, 546-551.

Wibmer, T., Rudiger, S., Heitner, C., Kropf-Sanchen, C., Blanta, I., Stoiber, K. M., Rottbauer, W., Schumann, C., 2013. Effects of Nasal Positive Expiratory Pressure on Dynamic Hyperinflation and Six Minute Walk Test in Patients with COPD. Respir. Care 699-708. doi:10.4187/respcare.02668

1. Apparatus for generating a supply of pressurized air at positive pressure for amelioration or treatment of a respiratory disorder, comprising:
a pressure generator configured to generate the supply of pressurised air for delivery to a delivery interface of a user;
at least one transducer configured to output at least one signal indicative of at least one respiratory characteristic of the user;
a controller, coupled with the pressure generator and at least one transducer, and configured to:
determine a cyclic pressure profile for the pressurised air;
control the pressure generator to achieve the pressure profile; and
determine, using the output of the at least one transducer, whether adjustment of the cyclic pressure profile is required.

2. The apparatus of claim 1, wherein the controller is configured to determine an initial set of therapy parameters for the cyclic pressure profile, the initial set of therapy parameters comprising one or more of: pressure during an inspiratory portion of the cyclic pressure profile, pressure during a expiratory portion of the cyclic pressure profile, a length of the inspiratory portion of the cyclic pressure profile, and a length of the expiratory portion of the cyclic pressure profile.

3. The apparatus of claim 2, wherein the determination of the initial set of therapy parameters is based on one or more of: user height, age, and weight.

4. The apparatus of claim 2, wherein the controller is configured to adjust the cyclic pressure profile based at least in part on determination of at least one trigger in a breath cycle of the user.

5. The apparatus of claim 4, wherein the controller is configured to distinguish triggers from false triggers.

6. The apparatus of claim 4, wherein the controller is configured to determine the at least one trigger based at least in part on a received indication of airway openness.

7. The apparatus of claim 6, wherein the indication of airway openness is conductance.

8. The apparatus of claim 6, wherein the controller is configured to determine the indication of airway openness based at least in part on a measure of flow rate, and a measure of pressure.

9. The apparatus of claim 1, wherein the controller is configured to adjust the pressure of the cyclic pressure profile based on one or more measured characteristics of the user's breath cycle, wherein the one or more measured characteristics are indicative of user effort.

10. The apparatus of claim 9, wherein the controller is configured to adjust the pressure of the cyclic pressure profile based on at least one of: a measure of average conductance, and peak conductance, over a plurality of breath cycles.

11. The apparatus of claim 1, wherein the controller is configured to adjust the cyclic pressure profile based on one or more predicted characteristics of the user's breath cycle.

12. The apparatus of claim 11, wherein the controller is configured to:
predict a length of a next inspiratory portion of the cyclic pressure profile, and a length of a next expiratory portion of the cyclic pressure profile, based on a measured length of a previous inspiratory portion of the user's breath cycle, and a measured length of a previous expiratory portion of the user's breath cycle; and
adjust the cyclic pressure profile based on the predicted length of the next inspiratory portion, and the predicted length of the next expiratory portion.

13. The apparatus of claim 1, wherein the cyclic pressure profile comprises a first changing portion comprising a rapid increase to a high pressure, a high pressure portion, a second changing portion comprising a slower decrease from the high pressure portion, and a low pressure portion.

14. The apparatus of claim 13, wherein the controller is configured to use a predicted length of an inspiratory portion of the cyclic pressure profile to control one or more of: timing of a transition between the high pressure portion and the second changing portion, and a rate of the decrease in pressure through the low pressure portion.

15. The apparatus of claim 13, wherein the controller is configured to use a predicted length of an expiratory portion of the cyclic pressure profile to control an increased flow rate near an end of the expiratory portion.

16. The apparatus of claim 13, wherein the controller is configured to control a transition between the second changing portion and the low pressure portion based at least in part of determination of at least one trigger.

17. The apparatus of claim 13, wherein the controller is configured to adjust the cyclic pressure profile on receiving a user input, wherein the adjustment of the cyclic pressure profile on receiving the user input comprises an adjustment of one or more of: a rate of increase in pressure through the first changing portion, a pressure level of the high pressure portion, and a hold time to maintain the high pressure portion.

18. The apparatus of claim 17, wherein the controller is configured adjust the cyclic pressure profile to maintain the high pressure portion for one of a plurality of hold times.

19. The apparatus of claim 18, wherein the controller is configured to select a hold time from the plurality of hold times based at least in part on whether the apparatus is in a continuous mode or an intermittent mode.

20. The apparatus of claim 19, wherein the controller is configured to select longer hold time in the intermittent mode than in the continuous mode.

21. The apparatus of claim 20, wherein the hold time in the continuous mode is between about 5 to 15% of an inspiratory portion of the cyclic pressure profile, and the hold time in the intermittent mode is between about 40 to 60% of the inspiratory portion of the cyclic pressure profile.

22. A system for amelioration or treatment of a respiratory disorder, comprising:
a delivery interface structured and configured to deliver a supply of pressurised air to a user;
the apparatus according to claim 1; and
an air circuit to connect the delivery interface and the apparatus, wherein the delivery interface is a mouthpiece interface.

23. A method for controlling cycling a supply of air at positive pressure for augmenting breathing of a user with shortness of breath, comprising:
determining in a controller a cyclic inspiratory pressure profile for a supply of pressurised air to be generated for delivery to a delivery interface device of the user;
controlling with the controller, generation of the supply of pressurised air with a pressure generator to achieve the inspiratory pressure profile; and
determining in the controller, using output of at least one transducer configured to output at least one signal indicative of at least one respiratory characteristic of the user, an adjustment of the inspiratory cyclic pressure profile.
